SEQUENCE LISTING (2 sequences)

SEQ ID NO: 1 - DNA, 52 bp, Artificial
  Substrate 1 used as a standard oligonucleotide with a fluorescent probe and a nucleotide bearing a propargyl-modified base, to confirm that ′click′ chemistry is functional with DNA molecules.
ggatccgaattcgcgctcgatcgcgcggatccgaattcgcgctcgatcgcgc

SEQ ID NO: 2 - DNA, 89 bp, Artificial
  Substrate 2 used as a standard oligonucleotide with a fluorescent probe, and a TaqI Mtase recognition site TCGA, to confirm that ′click′ chemistry is functional with DNA molecules.
ttaattaatcgaattgtaatacgactcactatagggagaggatccgaattcgcgctagatcgcgctagcgcggccgcgctgagcaataa

(19) United States
(12) Patent Application Publication
(10) Pub. No.: US 2013/0288264 A1
(43) Pub. Date: Oct. 31, 2013

(54) Development of a New Isolation Method with Higher Selectivity and Efficiency

(71) Applicant: The Regents of the University of California, Oakland, CA (US)

(72) Inventor: Youn-Hi Woo, San Ramon, CA (US)

(21) Appl. No.: 13/717,688

(22) Filed: Dec. 17, 2012

Related U.S. Application Data
(60) Provisional application No. 61/576,953, filed on Dec. 16, 2011.

(51) Int. Cl. C12Q 1/68

(52) U.S. Cl. CPC C12Q 1/6806
USPC 435/6.19

(57) ABSTRACT

A sample extraction method with improved sensitivity for capture of trace amounts of target by utilizing a target-specific enzyme, and covalently bonding the target for capture and immobilization on an activated surface.

### CROSS-REFERENCE TO RELATED APPLICATIONS

This application is a non-provisional utility application of and claims priority to U.S. Provisional Patent Application No. 61/576,953 filed on Dec. 16, 2011, which is hereby incorporated by reference in its entirety.

### STATEMENT OF GOVERNMENTAL SUPPORT

This invention was made with governmental support under the Department of Homeland Security Science and Technology directorate, Chemical and Biology division, Bioforensics Office (LB09005872). This work was supported by the Director, Office of Science, Office of Basic Energy Sciences, of the U.S. Department of Energy under Contract No. DE-AC02-05CH11231. A part of this work performed under the auspices of the U.S. Department of Energy by Lawrence Livermore National Laboratory under Contract DE-AC52-07NA27344. The government has certain rights in the invention.

### REFERENCE TO A SEQUENCE LISTING APPENDIX

A sequence listing is submitted concurrently with the specification and is part of the specification and is hereby incorporated in its entirety by reference herein.

### BACKGROUND OF THE INVENTION

1. Field of the Invention

The present invention is in the field of DNA isolation, extraction and purification methods useful in various research fields including forensics, food safety and diagnostics.

2. Related Art

Reliable and highly efficient DNA extraction methods have been a core element of various research fields including forensics and diagnostics. Tremendous efforts to develop an efficient method to extract and thereby isolate DNA with high sensitivity and selectivity from complex samples in a time saving manner have met with success. For example, silica-based resins or membranes integrated into a microchip for high-throughput DNA analysis have been particularly successful and are commonly used (R. Boom, C. J. A. Sol, M. M. M. Salimans, C. L. Jansen, P. M. E. W.-V. Dillen, And J. V. D. Noordaa, Rapid and Simple Method for Purification of Nucleic Acids. Journal Of Clinical Microbiology 28 (1990) 495-503). However, as DNA has become an important player in providing pivotal clues in research, forensics, and diagnostics, requirements of successful DNA extraction methods have been redefined, as well as those for subsequent analyses. Therefore, conventional methods for DNA extraction from complex samples, which mostly include a multistep process (cell lysis, breakdown of DNA-related proteins, and then isolation of DNA using various chemistries such as ethanol precipitation or selective binding of DNA to silica-based surface or glass fiber matrix), have been challenged due to lower compatibility with downstream analyses [H. Tian, A. F. R. Hu\ddot{}hmer, and J. P. Landers, Evaluation of Silica Resins for Direct and Efficient Extraction of DNA from Complex Biological Matrices in a Miniaturized Format. Analytical Biochemistry 283 (2000) 175-191]. That is, the majority of the commercially available clean-up kits are based on ‘Boom chemistry’ [R. Boom, C. J. A. Sol, M. M. M. Salimans, C. L. Jansen, P. M. E. W.-V. Dillen, And J. V. D. Noordaa, Rapid and Simple Method for Purification of Nucleic Acids. Journal Of Clinical Microbiology 28 (1990) 495-503], which relies on binding between nucleic acids and silica in the presence of high concentrations of chaotropes, such as 6-8 M guanidine or detergent. While these approaches prove sufficient for some applications, the current methods suffer several short-comings. First, due to the nonspecific separation mechanism based on ionic interaction, it mostly yields ‘not-so-perfectly pure’ final sample with other negatively charged polymers such as polysaccharides, which further complicates the downstream analyses. Second, they require high concentrations of salt as a chaotrope to disrupt the bio-membranes of the sample and mix up DNAs of different biological origins. Third, current commercial methods are designed primarily to treat the samples of smaller volumes (mL) to adapt to high-throughput procedures.

Ideally, a new DNA extraction method would combine improved purity and higher yields with a better compatibility with downstream analyses. The new technique would be highly selective for the DNA of interest in a complex sample and able to extract DNA in a non-invasive manner. In addition, it should be highly adaptable to a broad range of downstream analyses and easily adaptable to high-throughput systems, requiring effectiveness over a range of volumes from a few micro-liters to several liters.

Among DNA-specific enzymes, DNA methyltransferases (Mtase) catalyze transfer of methyl groups to DNA from a co-factor, S-adenosyl-L-methionine (SAM), upon recognition of short nucleotide sequences (4-7 nts). The mechanism of DNA Mtase and exploitation of the enzyme for numerous pharmaceutical purposes have been of particular interest [S. Bheemanai, Y. V. R. Reddy, And D. N. Rao, Structure, function and mechanism of exocyclic DNA methyltransferases. Biochemistry Journal 399 (2006) 177-190]. Most recently, chemically modified SAM was suggested to provide a chemical probing tool for DNA Mtase-related biochemical reactions [C. Dalhoff, G. Lukinavicius, S. Klimasauskas, and E. Weinhold, Direct transfer of extended groups from synthetic cofactors by DNA methyltransferases. Nat Chem Biol 2 (2006) 31-32, hereby incorporated by reference]. Dalhoff, et al., demonstrated that the TaqI methyltransferase from Thermus aquaticus uses a ‘propargyl group’ to modify the DNA instead of the natural methyl moiety from SAM. Ibid. Tremendous efforts to develop an efficient method to extract and thereby isolate DNA with high sensitivity and selectivity from complex samples in a time.

### SUMMARY OF THE INVENTION

The present invention describes systems and method that rely upon employing a target specific enzyme and using click chemistry between a modified target and the activated surface to pull down only the target in the sample. The covalent bond between the surface and the target enables the method to be highly selective and efficient and resulting in a highly purified target final sample.

The target in a sample can be any biological or chemical target molecule. In some embodiments, the target in the sample is DNA or RNA. In another embodiment, the target is a polysaccharide.

The strategy of the methods as applied to DNA is two-fold: (1) providing high selectivity and efficiency by employing a DNA specific enzyme, such as DNA methyltransferase and; (2) producing a highly purified DNA final sample and high compatibility with further treatments and downstream assays due to using click chemistry between modified DNA and the activated surface which pulls down only DNA in the sample.

For many applications, the samples to be analyzed are often from environment such that DNA of interest is buried in the cocktail of variety components, which makes efficient extraction of DNA quite challenging when a general separation technique is applied. Most of the commercially available DNA extraction methods, however, utilize either filtration through Si based-membrane or paramagnetic particles, through which the recovery yield may be compromised or not only DNA of interest but also other biopolymers can be co-extracted. Therefore, by employing the enzyme that will recognize only DNA in the mixture, we find the method to demonstrate much improved specificity toward DNA compared to the other commercial methods. Using the presently described method to pull down the DNA of interest via covalent bond without chemical or biological reagents also enables a broader range of cleanup processes without losing the recovered DNA. The well known ‘click’ chemistry enables the covalent bond formation between the activated surfaces and the DNA. ‘Click’ chemistry has been widely used both in the biological orthogonal reactions and chemical entities where seamless linking is highly desired without using other chemical reagents.

### BRIEF DESCRIPTION OF THE FIGURES

FIG. 1 is a schematic overview of the system and assay method as applied to DNA. (1) DNA of interest in complex environmental and biological samples; will be (2) modified via TaqI Mtase and chemically modified SAM; (3) Azide-activation of the selected surface; (3) DNA pull-down through ‘click’ chemistry; (4) Removal of the contaminants and subsequent downstream analyses.

FIG. 2A shows the standard oligonucleotide substrate sequences used. FIG. 2B shows the MTaqI reaction with alkynyl-SAM (Top image) or natural SAM (bottom image) followed by ‘click’ chemistry in the presence of Cu²⁺. The fluorescence-labeled DNA was pulled down onto the beads due to successful recognition between DNA and MTaqI, successful modification by Alkynyl-SAM.

FIG. 3 shows gel electrophoresis images which demonstrate that the modified site is tolerated by polymerase. (A) PCR product from the beads with no DNA (−), from the soluble free template (+), from the beads with DNA after ‘click’ chemistry (beads), the supernatant after final wash (S/N); (B) with and without recognition sites within the PCR products of different length.

FIG. 4 is an image of a gel showing that method's limit of detection: The Mtase method yielded PCR product of right size and sequence with DNA of as low as 0.1 fg (lane 1 and 2; negative control, lane 3 through 14; DNA of different concentrations (0.1 fg to 10 ng), lane 15; positive control).

FIG. 5 show the steps for modification of Si-beads with azide and fluorescence-labeled butyne.

FIG. 6 shows the RTaqI restriction digestion assay after Mtase treatment of the standard DNA.

FIG. 7 shows a gel images that demonstrate the efficiency of Mtase method in a broad range of pH and common salts.

FIG. 8 is a an overview of the method as highly DNA specific and efficient: via using DNA Mtase shows that final DNA sample with higher purity: via ‘click’ chemistry

FIG. 9 shows that the method is results in highly efficient DNA multi-extraction via a selective pull-down strategy. Selective pull-down strategy: more sensitive and efficient than conventional filtering method (Improved Sensitivity: pico-grams of DNA vs sub-fg of DNA). Highly compatible with downstream processes: DNA immobilization via covalent bond (e.g. on-chip PCR).

FIG. 10 shows an evaluation of the method compared to a commercially available kit.

FIG. 11. (A). Study on limit of detection. The Mtase method yielded PCR product of the right size and sequence using as low as 0.1 fg DNA template [lane 1, beads only without DNA; lane 2, supernatant from final wash; lane 3 through 14, DNA of different concentrations (from lanes 3 to 14: 0.1 fg, 1 fg, 10 fg, 100 fg, 1 pg, 10 pg, 100 pg, 500 pg, 1 ng, 10 ng, 100 ng, 500 ng); lane 15, soluble free DNA template]. The lanes between lane 3 and lane 4 and lane 11 and lane 12 are molecular markers showing the PCR product is ca. 400 bp long, as expected. (B) Efficiency of Mtase method in a broad range of pH (Top) and in the presence of common salts (Bottom). (Top) PCR products using the beads from different pH: (lane 1) pH 2; (lane 2) pH 3; Marker; (lane 3) pH 4; (lane 4) pH 5; (lane 5) pH 6; (lane 6) pH 7; (lane 7) pH 8; Marker; (lane 8) pH 9; (lane 9) pH 10; (lane 10) (+) control, free λ DNA; (lane 11) (−) control, the beads without DNA. (Bottom) PCR products using the beads with DNA extracted from high salt concentrations (lane 1) 10 mM; (lane 2) 1 mM; (lane 3) 100 nM; (lane 4) 10 nM CaCl₂; marker; (lane 5) 10 mM; (lane 6) 1 mM; (lane 7) 100 nM; (lane 8) 10 nM NaCl; marker; (lane 9) 10 mM; (lane 10) 1 mM; (lane 11) 100 nM; (12) 10 nM MgCl₂. (C) PCR products using the DNA-bound beads after Mtase-‘click’ chemistry. (Left) PCR products from the soil samples with 50 pg (lane 1), 500 pg (lane 2), 5 ng (lane 3), 50 ng (lane 4) of λ DNA, marker, and (+) control free λ-DNA (lane 5). (Right) PCR products using (lane 1) (+) control, purified DNA from E. coli (ATCC4157); (lane 2) (+) control, the supernatant (1 mL) after lysis; (lane 3) 50 μL; (lane 4) 150 μL; (lane 5) 300 μL; (lane 6) 500 μL; (lane 7) 700 μL, (lane 8) 1 mL of E. coli suspension samples after Mtase-‘click’ purification, and (lane 9) (−) control, sample without E. coli.

### DETAILED DESCRIPTION OF THE EMBODIMENTS

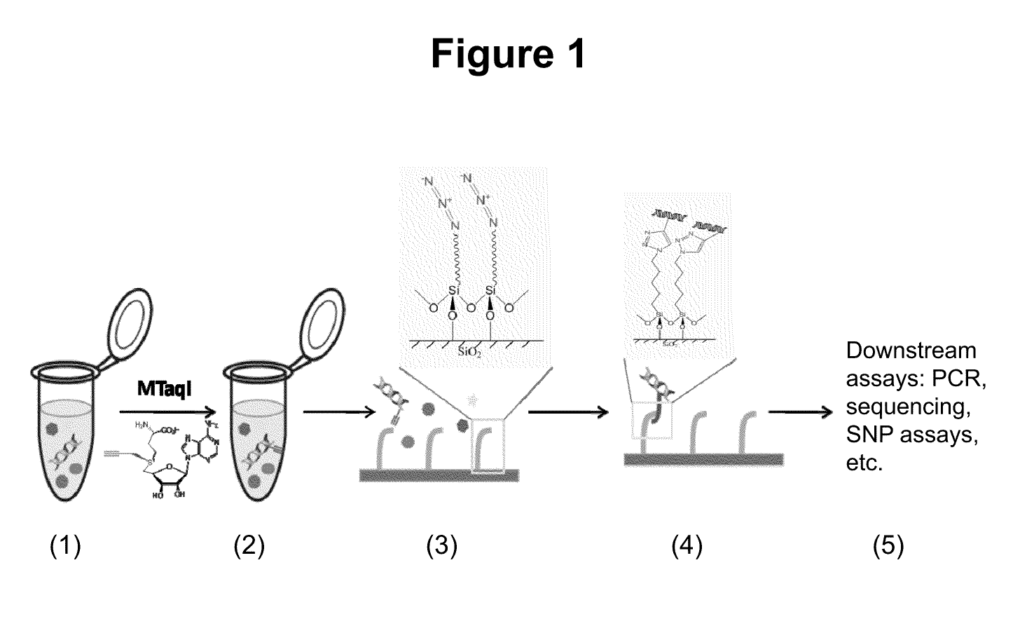

In one embodiment, compositions, systems and methods for employing a nucleotide-specific enzyme and an analogue of the cofactor S-adenosyl methionine (SAM) to modify nucleotide molecules and further immobilize them on an azide-modified surface through covalent bonding. FIG. 1 shows an overview of the designed compositions and assay method. After extensive washing, this method yields desired nucleotides with a greater purity without contaminating inorganic and organic species.

The present systems and methods rely upon employing a target specific enzyme and using click chemistry between a modified target and the activated surface to pull down only the target in the sample. The covalent bond between the surface and the target enables the method to be highly selective and efficient and resulting in a highly purified target final sample. The target in a sample can be any nucleotides, deoxyribonucleic acids (DNA), ribonucleic acids (RNA), proteins, peptides, receptors, antibodies, lipids, probes, ligands, small molecules, drugs, sugars, polysaccharides, cells, etc. In some embodiments, the target in the sample is DNA or RNA. In another embodiment, the target is a polysaccharide.

The strategy of the methods as applied to DNA is two-fold: (1) providing high selectivity and efficiency by employing a DNA specific enzyme, such as DNA methyltransferase and; (2) producing a highly purified DNA final sample and high compatibility with further treatments and downstream assays due to using click chemistry between modified DNA and the activated surface which pulls down only DNA in the sample.

Thus, in various embodiments, a method comprising (a) contacting DNA Mtase and a propargyl analogue of the cofactor SAM to a DNA molecule in a sample; (b) labeling the DNA molecule; (c) immobilization of the DNA molecule on an azide-modified surface through covalent bonding.

The method is highly compatible with different surfaces, substrates and platforms including but not limited to, beads, planar surfaces, or microfluidic chips for ultra-high throughput assays. The surface may be glass, silicon, metal or any polymer surface that may be functionalized to display an activated surface. In some embodiments, the surface may be glass or silica beads, polymer beads, magnetic beads, polymer coated magnetic beads, or coating on plastic tubing, microfluidic channels and chips.

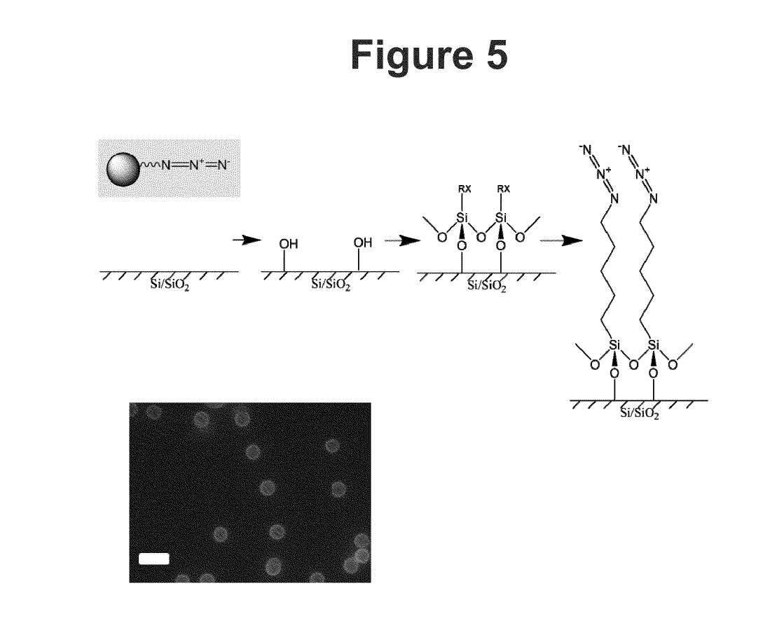

In various embodiments, the surface is activated or functionalized by attaching a monolayer of azide-terminated molecules. In various embodiments, the azides are attached to the surface through an attachment and linker region. The attachment of the azide to the substrate surface can be through a silane, siloxane, polydimethylsiloxane, carboxylate or amine or the like. In some embodiments, the attachment is through a silane or siloxane. FIG. 5 shows the steps for preparation of an azide activated Si surface. In some embodiments, the modification and functionalization is carried out as previously described in T. Lummerstorfer, and H. Hoffmann, J. Phys. Chem. B 108 (2004) 3963-3996, hereby incorporated by reference in its entirety.

In some embodiments, the linker region can be a carbon linker such as an alkane of 6 to 10, 15, 18 or up to 25 carbons in length. In other embodiments, the carbon linker region is 6 to 18 carbons in length.

In one embodiment, to facilitate release of the captured DNA, a cleavable crosslinker is included in an alkane linker region. In some embodiments, it is preferred that the cleavable crosslinker is close to the azide, thus about 2-5 carbons from the azide. In one embodiment, the cleavable crosslinker is about 2 carbons from the azide. In some embodiments, the crosslinker is a photocleavable crosslinker. In order to maintain the integrity of any captured DNA, the. photocleavable crosslinker should not require any initiation or activation by UV or non-visible light. Examples of visible light photocleavable crosslinkers include but are not limited to acylgermanes, and coumarins such as 7-amino coumarin based phototriggers.

After surface activation, the substrate surface is ready to interact with the sample. The sample may be any heterogeneous reaction mixture or sample including but not limited to such mixtures as biomass, crude lysate, cell culture, plant or organic matter, native glycans, environmental samples, biological samples, etc. Non-limiting examples of environmental samples that can be assayed in several embodiments described herein further include foods, water, plant matter, wood, leaves, paper waste, soil, compost, agriculture waste (e.g. livestock waste), mulch, dirt, clay, and garbage. Other examples of agricultural or biological samples include but are not limited to animal or human bodily fluids such as blood, urine, plasma, milk, or waste, or animal or plant food products including dairy, meat, eggs, poultry, fish, raw foods, etc.

The sample is subjected to general mechanical and/or dilutive sample pre-treatments such as centrifugation and dilution. This allows the serial dilution of the supernatant of any sample to allow for pull-down of DNA in the sample for testing with the present methods and systems without needing additional chemical modification.

The DNA in the sample is labeled using a DNA nucleotide-specific enzyme In some embodiments, the DNA nucleotide-specific enzyme is DNA Methyltransferase (Mtas)e because of the high specificity of DNA Mtase toward DNA. In some embodiments, the label is an analogue of the cofactor SAM is used. In various embodiments, a propargyl analogue of the cofactor SAM such as 2-butynyl-modified S-adenosyl methionine is used.

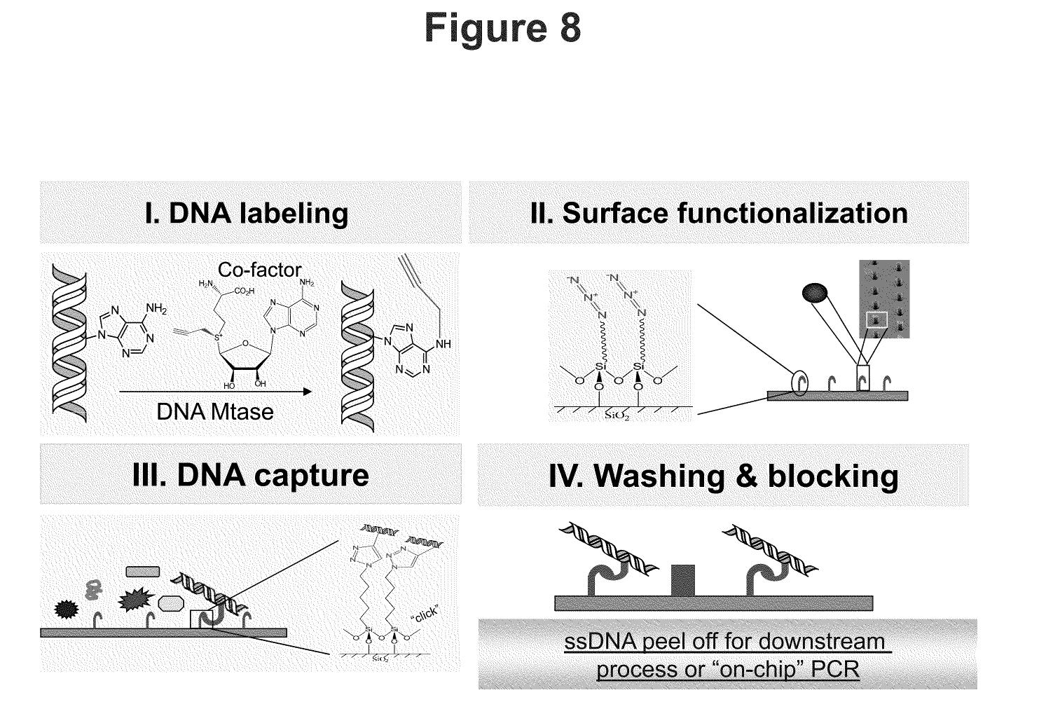

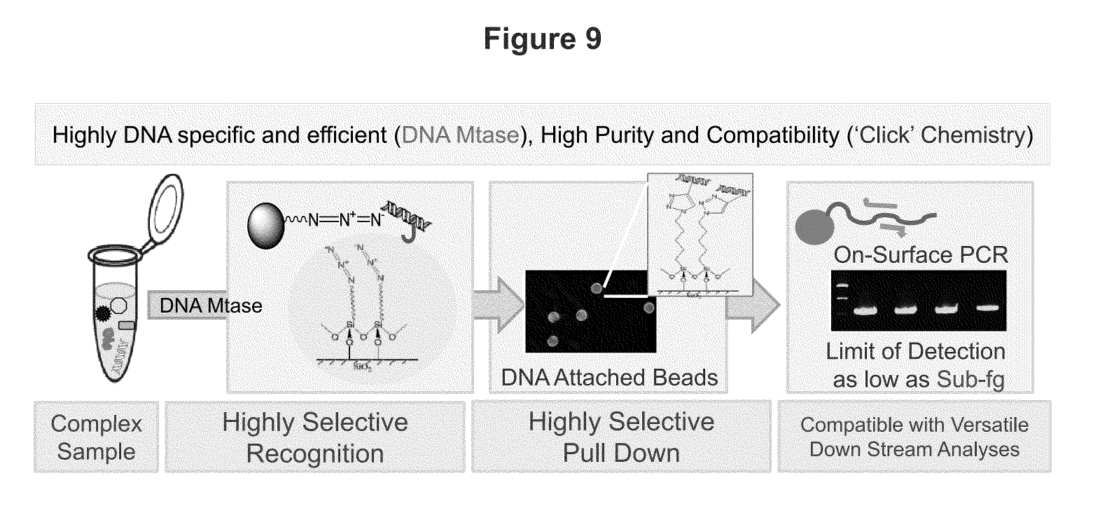

As shown in FIGS. 8 and 9, DNA in the sample is captured on the substrate surface and through the click chemistry reaction of azide alkyne Huisgen cycloaddition using a Copper (Cu) catalyst at room temperature as described by Barry Sharpless et al. in Rostovtsev, Vsevolod V.; Green, Luke G; Fokin, Valery V.; Sharpless, K. Barry (2002). “A Stepwise Huisgen Cycloaddition Process: Copper(I)-Catalyzed Regioselective “Ligation” of Azides and Terminal Alkynes”. Angewandte Chemie International Edition 41 (14): 2596-2599, hereby incorporated by reference. Since its first introduction by K. Barry Sharpless in 2001, ‘Click’ chemistry has received tremendous attention due to its ability to irreversibly couple two molecular modules under mild conditions. 1,3-dipolar cycloaddition between a propargyl group and an azide moiety, in particular, is a typical example of ‘click’ chemistry. This particular ‘click’ chemistry has been widely used for biological applications because both moieties are easily introduced into biomolecules and are stable under physiological conditions. Several groups have reported efficient immobilization of propargyl-carrying synthetic oligonucleotides onto the azide-modified surfaces with and without Cu(II) catalyst (D. I. Rozkiewicz, J. Gierlich, G. A. Burley, K. Gutsmiedl, T. Carell, B. J. Ravoo, and D. N. Reinhoudt, Transfer Printing of DNA by “Click” Chemistry. Chem. BioChem 8 (2007) 1997-2002; N. K. Devaraj, G. P. Miller, W. Ebina, B. Kakaradov, J. P. Collman, E. T. Kool, and C. E. D. Chidsey, Chemoselective Covalent Coupling of Oligonucleotide Probes to Self-Assembled Monolayers. Journal of American Chemical Society 127 (2005) 8600-8601). In those studies, chemically assembled short oligonucleotides were modified with propargyl groups on their bases before application to various azide-activated surfaces, such as gold, silica and biodegradable polymer fibers (Q. Shi, X. Chen, T. Lu, and X. Jing, The immobilization of proteins on biodegradable polymer fibers via click chemistry. Biomaterials 29 (2008) 1118-1126). All together, these studies clearly demonstrate the versatility of the matrices for the azide-activated surfaces and a broad range of application formats.

To exploit these advantages, ‘click’ chemistry is employed to allow the capture and immobilization of the labeled or modified DNA from the sample onto the activated substrate surface. Herein the azide is attached to the substrate surface and the alkyne is added to the DNA by a DNA MTase. FIG. 8(I) DNA labeling shows that an alkyne is added to the DNA by the DNA Mtase after incubation with a butynyl SAM analogue. Once the DNA nucleotides in the sample have been sufficiently modified to feature the alkyne, the DNA sample is contacted with the activated substrate surface and the reaction occurs thereby capturing the DNA to the substrate surface. In one embodiment, the DNA sample can be added or poured onto a planar substrate surface or in other embodiments, if the surface is a bead, the activated beads can be added to the DNA sample directly. In some embodiments, the baseline ratio of target DNA:active surface is about 1:1 M or vice versa.

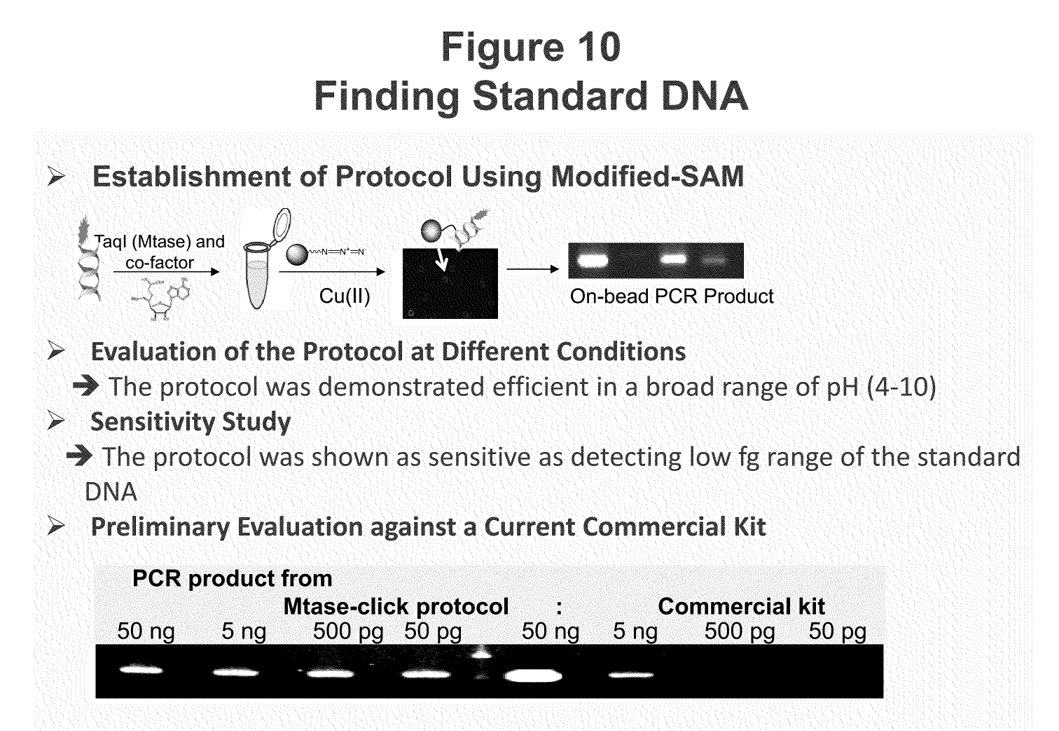

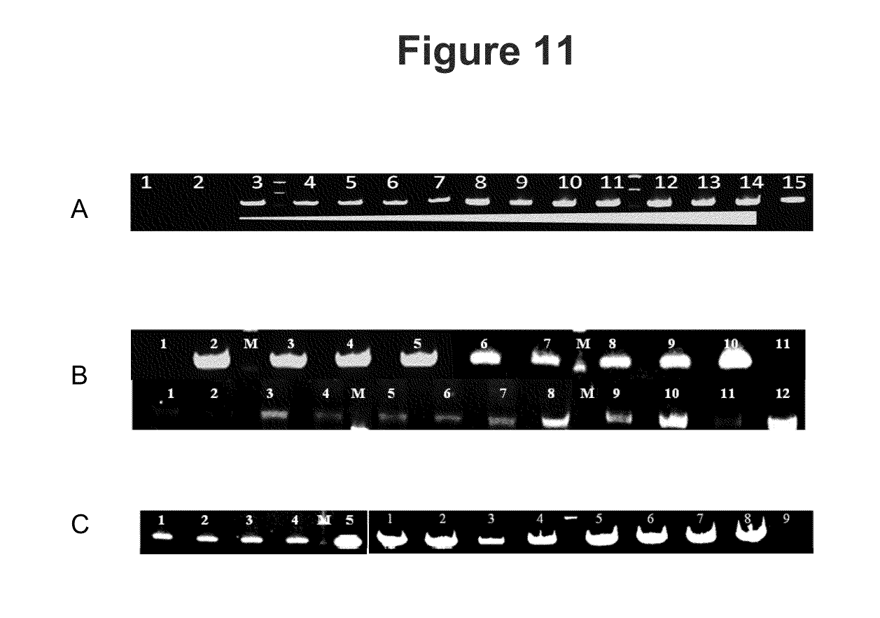

After allowing sufficient period for DNA capture, the supernatant is washed off the substrate surface leaving only captured DNA attached to the substrate surface. FIG. 10 shows the sensitivity of the present system and methods to efficiency of the pull-down assay to at least the pictogram level and to the sub-femtogram range (FIG. 11). The DNA can then be analyzed by various known downstream methods such as polymerase chain reaction, sequencing, single nucleotide polymorphism assays, etc.

It is contemplated that the method can also be applied to other types of targets such as protein to establish ‘one-pot, multi-extraction’ method. In such cases, other components of the sample are labeled with an alkyne using different enzyme that is specific for the component of interest.

In some embodiments, the present method and systems may be incorporated into a manufacturing or diagnostic process. For example, the present method may find use in the food industry whereby food samples are pre-treated and diluted, the DNA in the sample is labeled by an DNA Mtase and then the labeled DNA is contacted with an activated surface and DNA down to the pictogram level can be detected and isolated. This facilitates the isolation of very low levels of DNA from microbial pathogens or bacterial contamination and the subsequent identification of the pathogens through various other analytic methods or devices.

In another embodiment, a kit is provided and may comprise of any delivery system for delivering materials or reagents for carrying out a method of the invention. In the context of assays, such delivery systems include systems that allow for the storage, transport, or delivery of arrays or beads with probes, reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing microtubules or ampules of the relevant reaction reagents such as as TaqI Mtase, the alkyne-SAM cofactor and/or copper adduct, diluted to appropriate amounts and/or supporting materials for conducting assays of the invention.

The kit can also include reagents for downstream sample processing. In some embodiments, the reagents comprise reagents for the PCR amplification of sample nucleic acids including primers to amplify regions of a highly conserved sequence such as regions of the 16S rRNA gene. In still other embodiments, the reagents comprise reagents for the direct labeling of DNA, RNA, or other biological molecule. In further embodiments, the kit may include instructions for using the kit. In other embodiments, the kit includes a password or other permission for the electronic access to a remote data analysis and manipulation software program. Such kits will have a variety of uses, including environmental monitoring, diagnosing disease, monitoring disease progress or response to treatment, and identifying a contamination source and/or the presence, absence, or amount of one or more contaminants.

### Example 1

### Capturing DNA on a Surface with Click Chemistry

In the present example, we employed ‘click’ chemistry to immobilize the modified DNA. The method finds its first strength in the use of DNA specific enzyme, TaqI methyltransferase (Mtase) that is highly robust (K_{cat}/Kₘ=10⁶M⁻¹S⁻¹, compared to Tripsin K_{cat}/Kₘ=10³M⁻¹5⁻¹) and is extremely substrate-specific to DNA.

First, the planar and spherical surface with Si was chemically modified as previously described in T. Lummerstorfer, and H. Hoffmann, J. Phys. Chem. B 108 (2004) 3963-3996, previously incorporated by reference in its entirety.

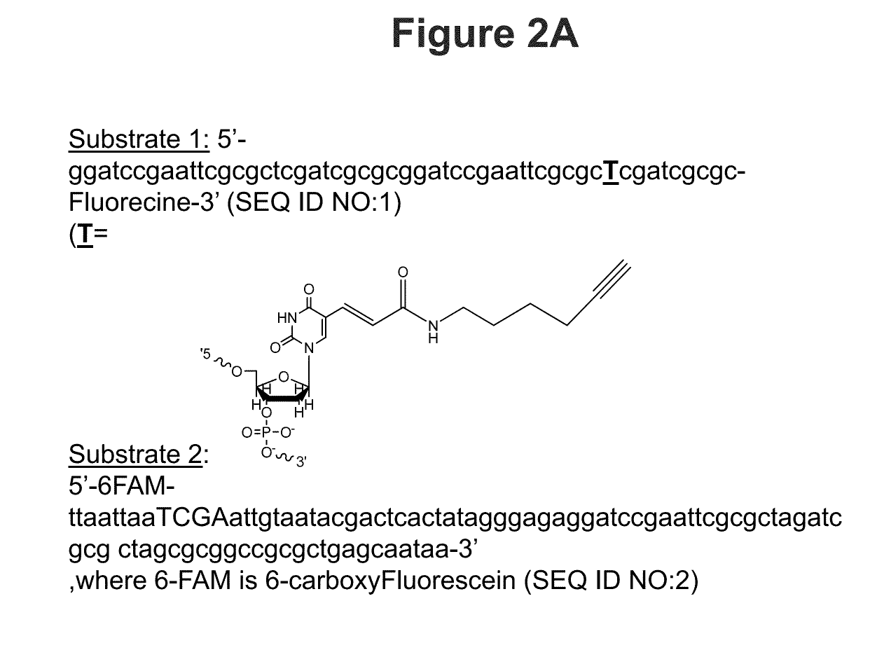

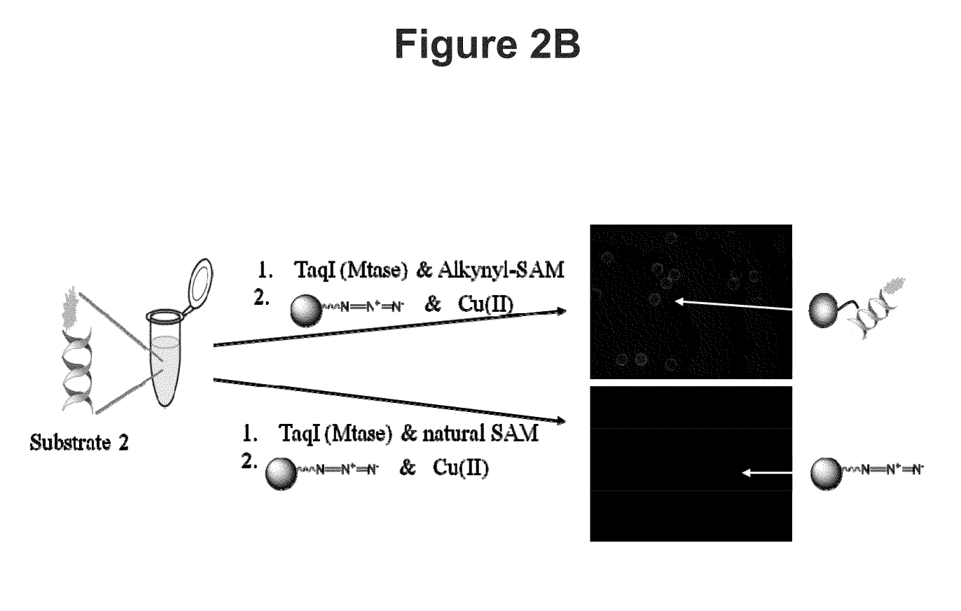

Successful modification of the surface with an azide (N₃) group was confirmed by adding the propargyl-amine, and subsequent fluorescent labeling (FIG. 5). We then designed two standard oligonucleotides (100 nucleotides) with a fluorescent probe, one with a nucleotide bearing a propargyl-modified base (substrate 1, FIG. 2A) and the other with TaqI Mtase recognition site TCGA, (substrate 2, FIG. 2A), to confirm that ‘click’ chemistry is functional with DNA molecules. Referring now to FIG. 1, in one embodiment, sequences of standard oligonucleotide substrates may be for Substrate 1: 5′-ggatccgaattcgcgctcgatcgcgcggatccgaattcgcgcTcgatcgcgc-Fluorecine-3′ (SEQ ID NO:1), where T is a propargyl-modified base, and for Substrate 2: 5′-6FAM-ttaattaaTCGAattgtaatacgactcactatagggagaggatccgaattcgcgctagatcgcgctagcgcggccgcgctgagcaataa-3′, where 6-FAM is 6-carboxyFluorescein (SEQ ID NO:2).

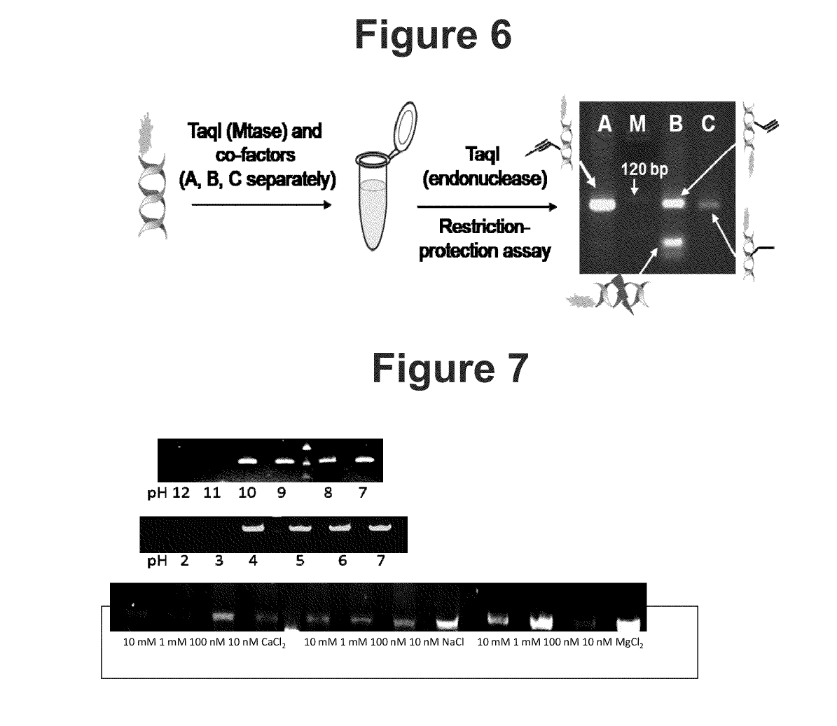

After the ‘click’ chemistry procedure, standard substrate 1 was successfully isolated as evidenced by fluorescently-labeled Si-beads. The substrate 2 was then treated with TaqI Mtase in the presence of 2-butynyl-modified S-adenosyl methionine (butynyl-SAM) (C. Dalhoff, G. Lukinavicius, S. Klimasauskas, and E. Weinhold, Direct transfer of extended groups from synthetic cofactors by DNA methyltransferases. Nat Chem Biol 2 (2006) 31-32) at 65° C. for 2 hrs before incubation with CuSO₄ in ascorbic acid solution (1 mM) (D. I. Rozkiewicz, J. Gierlich, G. A. Burley, K. Gutsmiedl, T. Carell, B. J. Ravoo, and D. N. Reinhoudt, Transfer Printing of DNA by “Click” Chemistry. Chem. BioChem 8 (2007) 1997-2002). Images from a fluorescence microscope show that substrate 2 was successfully bound to the azide-activated beads only in the presence of both TaqI Mtase (MTaqI) and butynyl-SAM (FIG. 2B) while the sample with natural co-factor SAM did not yield fluorescing beads, proving only the oligonucleotides modified with butynyl-SAM were associated with the beads. The restriction-protection assay (FIG. 6) was used to investigate MTaqI efficiency with a series of chemically modified cofactors, including butynyl-SAM, propargyl-SAM, and the natural cofactor SAM, where butynyl-SAM showed as good efficiency as the natural cofactor, SAM.

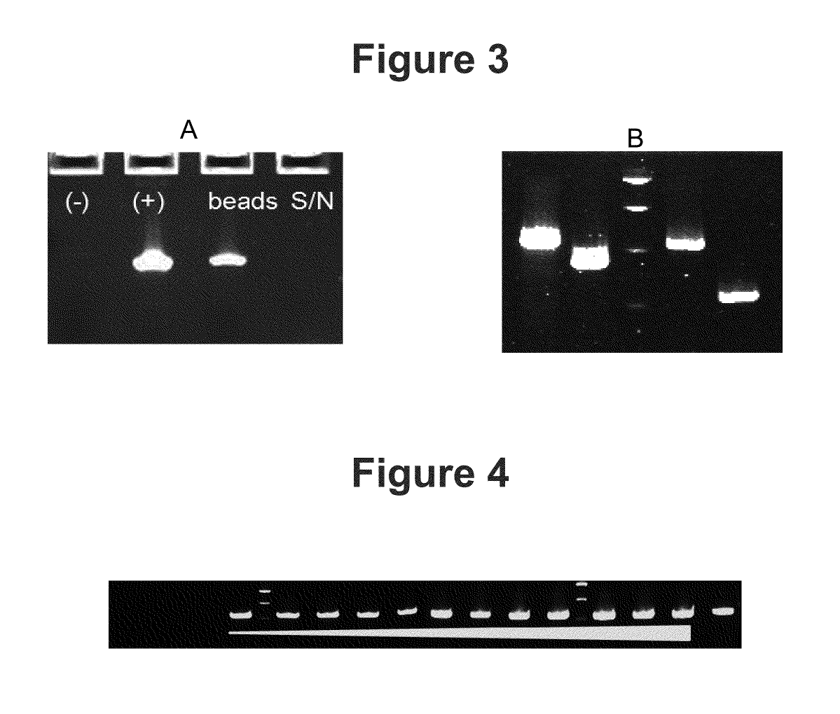

Once it was confirmed that the enzymatically modified DNA was successfully bound to the azide-activated Si surface, we used the beads as a template for ‘on-bead PCR’. Using Dam(−) λ-DNA (linear DNA with 48,502 bp, contains 121 recognition sites for TaqI Mtase, NEB, Ipswich, Mass.), we performed the MTaqI reaction followed by ‘click’ chemistry with azide-modified Si beads. Each supernatant from washing steps were subjected to PCR to investigate presence of un-bound DNA. Further quantification after ‘click’ chemistry using spectrophotometer suggested that immobilization was approximately 90% complete (<10% loss of sample DNA). This result demonstrates that the Mtase method provides an alternative to the current methods with much improved yield over current membrane-based methods a common drawback of which is low yield due to non-specific binding. It should be noted that the λ-DNA modified with 2-butynyl-SAM was tolerated by the selected polymerases including Taq, Deep Vent, and Pfu DNA polymerase to yield the PCR products of right sizes and sequences (FIG. 3).

After determining the yield, we investigated sensitivity of the method. Due to the high specificity between DNA and Mtase, the method is expected to capture DNA as small as 15 base pairs. Using serially diluted standard DNA (10 ng to 0.1 fg), we performed the Mtase method and ‘click’ chemistry before subjecting the beads to PCR. All of the resulting beads were used for PCR to measure DNA detection limit. The Mtase method yielded PCR product from all the beads of different DNA concentrations as low as 0.1 fg (FIG. 4). Compared to the currently available DNA extraction kits, most of which show sensitivity in terms of nanograms of DNA, this result shows that the Mtase method enables detection of DNA with single digit copy number. Another requirement of a successful new assay should be robustness of the procedure in various sample conditions. Therefore, we have investigated efficiency of the Mtase method in a broad range of pH and salt concentrations. The PCR product after Mtase and ‘click’ chemistry showed that the Mtase method is effective in the range of pH 4-10 and in the presence of nM to mM concentrations of common salts such as Ca²⁺, Mg²⁺, and Na⁺ (FIG. 7.).

The chemicals and organic solvents were purchased from Sigma-Aldrich unless indicated otherwise. SAM was chemically modified and purified using C₁₈ HPLC as a diastereo-mixture [4]. The oligonucleotides used as standard substrates were purchased from Integrated DNA Technology (IDT, Coralville, Iowa). The silica beads were purchased from Bangs Laboratory (Fishers, Ind.). TaqI methyltransferase (MTaqI), TaqI endonuclease (RTaqI), and Dam(−) λ DNA were purchased from New England Biolabs (Ipswich, Mass.). TaqI methyltransferase is expressed as the poly-His form in an E. coli system and purified using standard molecular biology and biochemistry techniques. PCR-related products were purchased from Invitrogen (Carlsbad, Calif.).

### Synthesis and Purification of 2-butynyl-SAM

Poly(4-vinylpyridine) (2.52 g, 24.0 mmol of pyridine residues) was added to dry CH₂Cl₂ (15 mL) before cooling the suspension to 0° C. with ice bath and addition of trifluoromethanesulfonic anhydride (6.21 g, 22.0 mmol) under Ar gas. Solution of 2-butynyl-1-ol (1.5 ml, 20 mmol) in dry CH₂Cl₂ (10 mL) was added drop-wise to this stirred suspension under Ar gas within 20 min. After stirring at room temperature for 10 min, poly(4-vinylpyridine) was filtered and washed with CH₂Cl₂ and the organic layer was concentrated. The solution of S-adenosyl-L-homocysteine (20 mg, 52 μmol) in a 1:1 mixture of formic acid and acetic acid (1 mL) was cooled in ice bath before addition of the activated butynyl-moiety from above. The reaction was quenched after 2 h by adding water (20 mL). The aqueous phase was washed with ether, concentrated, and freeze dried over night. The next day, the sample was dissolved in 3 mL of water and purified using C18 reverse phase HPLC system eluting with methanol (linear gradient to 100% in 15 min) in aqueous ammonium formate (20 mM, pH 3.5) by monitoring at 260 nm. Purified fractions were pooled, freeze-dried, and characterized by ES-MS (M⁺=423 for propargyl-AdoMet, M⁺=437 for 2-butynyl-AdoMet).

### Preparation of Azide Modified Si-beads

The SiO₂ beads (2-μm, Bangs Lab, Fishers, Ind.), previously rinsed with ethanol, water, and CH₂Cl₂, were resuspended in 1 mL 1% (v/v) 1-bromoundecyltrichlorosilane in CH₂Cl₂ and incubated at room temperature for 1 h before rinsing with CH₂Cl₂, ethanol, and DMF. The beads then were incubated in 1 mL of saturated NaN₃ solution in DMF at room temperature overnight followed by rinsing with DMF, ethanol, and water.

### TaqI Mtase Reaction

λ-DNA (5 ng/μL), Triton X-100 (0.01%), BSA (0.1 mg/mL), 2-butynyl-SAM (50 μM), and MTaqI (0.5 U) were mixed in total volume of 40 μL of NEB buffer #4 before incubation at 60° C. for 2 hr.

### Restriction-Protection Assay Using RTaqI

After Mtase reaction, 1 μL of RTaqI (NEB, Ipswich, Mass.) was added to an aliquot (20 μL) of the Mtase reaction mixture and incubated at 60° C. for 30 min. The protection of the corresponding DNA by Mtase in the presence of natural and chemically modified co-factor SAM was monitored using 0.8% agarose gel.

### ‘Click’ Chemistry Between Alkyne-Modified DNA and Azide-Activated Beads

The Mtase reaction mixture from above (total 40 μL) was mixed with 20 μL of azide-modified bead suspension in total volume of 100 μL of ascorbate solution (containing 1 mg of sodium ascorbate and 0.5 mg of disodium bathophenanthroline disulfonate in 5 mL of water) at room temperature overnight. The next day, the beads were rinsed with H₂O and ethanol before resuspension into 10 μL of PBS.

### ‘On-Bead’ PCR

Two μL of the suspension above was used as template for a PCR reaction by Taq polymerase (95° C. for 4 min; denaturation at 95° C., 1 min; annealing at 55° C., for 1 min; elongation at 72° C. for 1.5 min; 35 cycles from step 2 to 4; final elongation at 72° C. for 10 min, then keep at 4° C.). The PCR product was identified on 0.8% agarose gel and sequenced to confirm tolerance of the modified sites by the polymerase.

Currently, development of reliable new methods to extract DNA from various conditions is urgently needed in various fields of research due to limitations of the popular membrane-based filtration or paramagnetic particle based-separation methods. Moreover, new methods specifically designed to treat small-volume samples in a ‘fast’ and ‘high-throughput’ manner are required. This Mtase method provides a new platform for DNA extraction to address these challenges. First, the Mtase method can be applied to any volume, offering an alternative for samples of larger volumes. Second, the Mtase method has demonstrated high sensitivity and selectivity for DNA as low as an fg of DNA. Third, the Mtase method can be combined with a broader range of downstream analyses due to purity of the sample. Further optimization of the protocol for different samples types and development of various surface formats such as magnetic beads and incorporation into the plastic tube surface will increase the range of application and utility of this assay.

### Example 2

### Capturing DNA from Soil Samples

We also have demonstrated that the method proved efficient in a broad range of sample conditions i.e.: (1) the ‘Mtase Click’ method developed in Example 1 works properly in the pH range of 4 to 10 without compromising its efficiency [FIG. 11(B, Top)]; (2) the method can tolerate broad range of salt variations without interfering PCR productivity with exception of higher concentrations (1 mM and 10 mM) of CaCl₂ [FIG. 11(B, Bottom)]. To determine its efficiency, we evaluated the Mtase method with samples of DNA mixed with soil and E. coli lysate. The soil samples were first prepared with the known amount of the standard λ DNA to demonstrate that the free-floating DNA in complex samples is selectively extracted despite the presence of concomitant components such as large particles, metals, and other bio-polymers, e.g. proteins, contained in soil.

As shown in FIG. 11(C, left), the free exogenous DNA in the sample was successfully extracted and subjected to PCR, giving the amplified product of the right size and sequence. We then extracted endogenous DNA from a cultured E. coli sample where the bacterial membrane was disrupted by freeze-thaw and bead beating. We confirmed that the MTase method functions effectively as evidenced by PCR products of the right size and sequence [FIG. 11(C, Right)]

The present examples, methods, procedures, specific compounds and molecules are meant to exemplify and illustrate the invention and should in no way be seen as limiting the scope of the invention. Any patents, publications, publicly available sequences mentioned in this specification are indicative of levels of those skilled in the art to which the invention pertains and are hereby incorporated by reference to the same extent as if each was specifically and individually incorporated by reference.

### REFERENCES

[1] R. BOOM, C. J. A. SOL, M. M. M. SALIMANS, C. L. JANSEN, P. M. E. W.-v. DILLEN, and J. V. D. NOORDAA, Rapid and Simple Method for Purification of Nucleic Acids. JOURNAL OF CLINICAL MICROBIOLOGY 28 (1990) 495-503.
[2] H. Tian, A. F. R. Hu\ddot{}hmer, and J. P. Landers, Evaluation of Silica Resins for Direct and Efficient Extraction of DNA from Complex Biological Matrices in a Miniaturized Format. Analytical Biochemistry 283 (2000) 175-191.
[3] S. BHEEMANAI, Y. V. R. REDDY, and D. N. RAO, Structure, function and mechanism of exocyclic DNA methyltransferases. Biochemistry Journal 399 (2006) 177-190.
[4] C. Dalhoff, G. Lukinavicius, S. Klimasauskas, and E. Weinhold, Direct transfer of extended groups from synthetic cofactors by DNA methyltransferases. Nat Chem Biol 2 (2006) 31-32.
[5] D. I. Rozkiewicz, J. Gierlich, G. A. Burley, K. Gutsmiedl, T. Carell, B. J. Ravoo, and D. N. Reinhoudt, Transfer Printing of DNA by “Click” Chemistry. Chem. BioChem 8 (2007) 1997-2002.
[6] N. K. Devaraj, G. P. Miller, W. Ebina, B. Kakaradov, J. P. Collman, E. T. Kool, and C. E. D. Chidsey, Chemoselective Covalent Coupling of Oligonucleotide Probes to Self-Assembled Monolayers. Journal of American Chemical Society 127 (2005) 8600-8601.
[7] Q. Shi, X. Chen, T. Lu, and X. Jing, The immobilization of proteins on biodegradable polymer fibers via click chemistry. Biomaterials 29 (2008) 1118-1126.
[8] T. Lummerstorfer, and H. Hoffmann, J. Phys. Chem. B 108 (2004) 3963-3996.

What is claimed is:

1. A method of isolating a target from a sample comprising the steps of: (a) contacting a target-specific enzyme and a labeled cofactor to a target molecule in a sample; (b) modifying the target with the label by the enzyme; (c) contacting the labeled target with a functionalized surface; (d) capturing the labeled target molecule on the functionalized surface through covalent bonding.

2. The method of claim 1 wherein the target-specific enzyme is a DNA methyltransferase and the target molecule is DNA.

3. The method of claim 1 wherein the cofactor is propargyl analogue of the cofactor SAM featuring an alkyne group.

4. The method of claim 1, wherein the surface is glass, silicon, or a polymer surface.

5. The method of claim 4 wherein the functionalized surface has an azide-terminated molecule attached thereto.

6. The method of claim 5, wherein the functionalized surface is azide-terminated molecule is attached to the functionalized surface through an attachment and linker region.

7. The method of claim 6, wherein the attachment is through a silane, siloxane, carboxylate or amine.

8. The method of claim 7, wherein the linker region is an alkyl group 6-18 carbons in length.

9. The method of claim 1, wherein the label is an alkyne.

10. The method of claim 1, further comprising the step (e) of washing off the sample leaving only the captured target molecule on the functionalized surface.

11. A method comprising (a) contacting DNA Methyltransferase (DNA Mtase) enzyme and alkyne-modified S-adenosyl methionine (alkyne SAM) cofactor to a DNA molecule in a sample; (b) labeling the DNA molecule with an alkyne, wherein the label is added to the DNA by the DNA Mtase enzyme; (c) contacting the sample containing the alkyne-labeled DNA with a azide-functionalized surface; (d) immobilizing of the DNA molecule on the azide-functionalized surface through reaction of the alkyne and the azide-functionalized surface.

12. The method of claim 11 wherein the alkyne SAM is 2-butynyl-modified S-adenosyl methionine.
